(19) United States
(12) Patent Application Publication
(10) Pub. No.: US 2003/0198679 A1
(43) Pub. Date: Oct. 23, 2003

(54) Flocculated pharmaceutical suspensions and methods for actives

(76) Inventors: Subhas C. Kundu, Ellicott City, MD (US); Vivek Desai, Ellicott City, MD (US); Andrea Cameron, Baltimore, MD (US)

Correspondence Address: DICKSTEIN SHAPIRO MORIN & OSHINSKY LLP, 2101 L STREET NW, WASHINGTON, DC, 20037-1526, US

(21) Appl. No.: 10/440,200

(22) Filed: May 19, 2003

Related U.S. Application Data
(63) Continuation of application No. 09/707,793, filed on Nov. 8, 2000, now abandoned.

(51) Int. Cl. A61K 31/715; A61K 31/56; A61K 9/26; A61K 9/14

(52) U.S. Cl. 424/489; 514/54; 514/169

(57) ABSTRACT

An aqueous pharmaceutical composition suitable for oral delivery has an insoluble active substance and one or more wetting agents in liquid suspension. The composition contains floccules of the active ingredient and is substantially free of polyethelyne glycol, propylene glycol, glycerol and sorbitol. The formulation has an excellent shelf-life in which caking and sedimentation are inhibited. The composition may be resuspended upon light to moderate shaking.

### FIELD OF THE INVENTION

The present invention relates to aqueous suspensions of active substances, and in particular, to aqueous flocculated suspensions containing one or more insoluble actives which are suitable for oral delivery. The invention also relates to the use of certain surfactants to enhance flocculation in aqueous pharmaceutical suspensions.

### BACKGROUND OF THE INVENTION

There have been many attempts to formulate aqueous suspensions of water-insoluble pharmaceutical active ingredients. Flocculated suspensions in particular are desirable in numerous applications. They are well suited for oral delivery of the active, and are often preferred for patients for whom swallowing pills or other dosage forms is difficult.

A flocculated suspension contains the active pharmaceutical dispersed throughout the liquid medium. Minute particles of the active agent associate themselves with one or more excipients to form an agglomerated mass which is referred to as a “floccule” or “floc.” Other excipients in turn act to suspend the snowflake-like flocs in the water. The goal is to achieve a dispersion in which the active pharmaceutical component can be uniformly suspended and dispersed upon light to moderate shaking. In this way, the patient can be assured of receiving not only the appropriate dosage of the active, but substantially the same dosage upon each administration.

Many surfactants available in the art act as wetting agents for water-insoluble actives. These wetting agents greatly facilitate the formation of aqueous suspensions by reducing die surface tension between the active and the aqueous phase. Other compounds function as suspending agents which maintain the wetted active in uniform dispersion throughout the liquid media. The problem which arises is finding the right combination of compounds which are best suited for the particular active. Another problem is finding the particular concentration range which will enhance flocculation and ensure adequate and uniform resuspendability and floccule size. In addition to achieving good dispersion and uniformity, another goal is ensuring the optimal bioavailability of the active. The floccules should permit the active to be absorbed by the body at a rate and in an amount which will facilitate its efficacy. Moreover, the active should be stable in the aqueous suspension over its entire shelf-life.

Atzinger et al., U.S. Pat. No. 5,338,732, is directed to a flocculated suspension containing the active substance megestrol acetate, together with both polyethylene glycol and polysorbate, in particular polysorbate 80. The requisite polyethylene glycol (PEG) is present in relatively large amounts in excess of 5% and as much as 30%. The polysorbate component is present in an amount of 0.005% to 0.015%. At polysorbate 80 concentrations as low as 0.025% the patentees note significant deflocculation and caking of the formulation. In addition, Table 4 in the reference shows a significant decrease in physical stability at a concentration of 0.02% polysorbate 80.

Ragunathan et al., U.S. Pat. No. 6,028,065, is also directed to a flocculated suspension containing megestrol acetate. Like the formulation set forth in Atzinger et al., the composition is described by the patentees as containing polyethylene glycol. More specifically, the patentees state that at least one compound selected from the group consisting of polyethylene glycol, propylene glycol, glycerol, and sorbitol is “critical” to the suspendability of megestrol acetate in a flocculated suspension. In addition, Ragunathan et al. specify that their formulation not contain polysorbate when polyethylene glycol is present.

Thus, there exists a need in the art to find one or more wetting agent compounds which together with one or more pharmaceutical actives can form a stable flocculated liquid suspension. There also exists a need for an improved flocculated suspension containing one or more actives together with a synergistic amount of one or more excipients. There is a further need in the art to minimize or avoid the use of polyethylene glycol and certain other similar compounds in forming a liquid formulation for delivery of an active.

### SUMMARY OF THE INVENTION

The invention according to one embodiment is a composition containing at least one insoluble active substance together with at least one wetting agent. The concentration of the wetting agent is sufficient to form a stable, flocculated suspension of the active substance.

Also provided as part of the invention is a method for forming a composition which involves combining at least one active substance and at least one wetting agent, wherein the wetting agent is present in an amount sufficient to form a stable, flocculated suspension of the active substance. The wetting agent may desirably be chosen from the group of docusate sodium and ethylene oxide/propylene oxide copolymers (including block copolymers).

The invention also provides an oral pharmaceutical composition having about 0.5 to about 10% of megestrol acetate; about 0.005 to about 1% of at least one wetting agent selected from the group of docusate sodium and ethylene oxide/propylene oxide copolymers and block copolymers, sorbitan fatty acid esters, polyoxyethylene sorbitan fatty acid esters, polyoxyethylene fatty acid esters, and about 0.01 to about 1.0% of at least one suspending agent.

The composition of the invention according to its various embodiments should desirably not contain any of the following excipients: polyethylene glycol, propylene glycol, sorbitol and glycerol. The present inventors have discovered for the first time effective flocculated pharmaceutical suspensions which do not require any of the above-referenced excipients. This discovery is particularly surprising in view of the prior art, including the Atzinger et al. and Ragunathan et al. patent referred above.

As part of the invention, there is also a method of forming an oral pharmaceutical composition in which xanthan gum and water are combined in a first vessel. A wetting agent such as docusate sodium, an ethylene oxide/propylene oxide copolymer, or both, and megestrol acetate are combined in a second vessel. The contents of the first vessel are then combined with the contents of the second vessel.

Additional advantages and features of the present invention will become more readily apparent from the following detailed description which illustrates various embodiments of the invention.

### DETAILED DESCRIPTION OF THE PREFERRED EMBODIMENTS

The pharmaceutical composition of the invention is described as a flocculated aqueous suspension. A suspension is one in which solid particles of one or more active substances are suspended within a liquid medium. The liquid medium may contain various excipients, especially one or more wetting/dispersing agents. These excipients maintain the active in combinations or aggregations of suspended particles known as “floccules” or “flocs” within the suspension.

The composition of the invention is also described as being “stable.” A stable suspension is one which can be redispersed or resuspended with light to moderate shaking throughout its shelf-life, thereby resisting caking or sedimentation. In addition, a stable suspension is one which resists changes in floccule particle size and distribution, the suspended active agent is not substantially degraded, nor is its bioavailability substantially affected over the course of its shelf life. The composition of the invention according to the embodiments hereinafter described should be stable, i.e., have a shelf-life of at least about two to about three months, preferably at least about 1 year, and more preferably at least about 18 months. It is especially desirable that the formulation be stable for at least about 2 to about 3 years, or even longer. Storage stability is typically measured with respect to ambient relative humidity, which is generally within the range of about 50% to about 80%, as well as temperature, which is typically within the range of about 25° C. to about 40° C.

In general, the aqueous pharmaceutical suspensions in accordance with the present invention will include an amount of at least one water-insoluble, pharmaceutically active agent which is sufficient to treat a mammal in need of treatment with the active. As used herein, the terms “water-insoluble” and “insoluble” refer to those substances which are insoluble, practically insoluble, or only slightly or sparingly soluble in aqueous media as those terms are described in the United States Pharmacopeia; Remington's Pharmaceutical Sciences, 18^{th} edition published by Mack Publishing Company.

The pharmaceutical active utilized in the invention is preferably micronized or pulverized so that it has a “dry,” e.g., non-wetted, mean particle diameter less than or equal to about 20 microns. Preferably, the mean particle diameter of the active substance alone will be within the range of about 1 micron to about 10 microns.

A non-exhaustive listing of suitable pharmaceutical actives from which the water insoluble active ingredient may be chosen include anti-cancer agents, antitussives, antihistamines, decongestants, alkaloids, mineral supplements, laxatives, vitamins, antacids, ion exchange resins, anti-cholesterolemics, anti-lipid agents, antiarrhythmics, antipyretics, analgesics, appetite suppressants, expectorants, anti-anxiety agents, anti-ulcer agents, anti-inflammatory substances, coronary dilators, cerebral dilators, peripheral vasodilators, anti-infectives, psycho-tropics, antimanics, stimulants, gastrointestinal agents, sedatives, antidiarrheal preparations, anti-anginal drugs, vasodialators, anti-hypertensive drugs, vasoconstrictors, migraine treatments, antibiotics, tranquilizers, anti-psychotics, antitumor drugs, anticoagulants, antithrombotic drugs, hypnotics, anti-emetics, anti-nauseants, anti-convulsants, neuromuscular drugs, hyper- and hypoglycemic agents, thyroid and antithyroid preparation, diuretics, antispasmodics, uterine relaxants, mineral and nutritional additives, antiobesity drugs, anabolic drugs, erythropoietic drugs, antiasthmatics, cough suppressants, mucolytics, anti-uricemic drugs, anti-viral drugs and mixtures thereof.

Of the foregoing, anti- anorexia, cachexia compounds are particularly preferred. Especially desirable is megestrol acetate. Megestrol acetate is the generic name for 17-α-acyloxy-6-methylpregna-4,6diene-3,20-dione. Megestrol acetate in oral suspension form has now been indicated for use as an appetite stimulant, particularly for those suffering from “wasting” afflictions as a result of cancer, or diseases of the immune system such as AIDS. Megestrol acetate is insoluble in water, and exhibits a considerable degree of hydrophobicity.

The amount of pharmaceutical active used in the invention will depend on various factors, including the sex, age, weight, general health and condition of the patient and the type of drug and suspension. As a general rule, from about 0.1% to about 25% by weight of at least one substantial water-insoluble pharmaceutical active agent will be used (the weight percentage for the active agent is provided herein on a weight to volume, or w/v basis, and unless otherwise stated, all other weight percentages provided herein are on a weight to weight, or w/w basis). It is possible, however, depending on the nature of the dosage form, the active(s), and the indication(s), to create suspensions in accordance with the present invention that have greater than about 20% or less than about 0.1% of the active substance.

More preferably, the amount of insoluble active agent included in the suspensions of the present invention will range from about 0.1% to about 10%. Suspensions containing about 1% to about 8% of the active are even more preferred, with amounts within the range of about 2% to about 6% being most preferred. Especially desirable is a concentration level of about 4% of the active substance. Megestrol acetate utilized at about 4% is particularly preferred for use as an active agent herein.

The flocculated suspensions in accordance with the present invention are principally prepared by combining the active substance with at least one wetting agent. The wetting agent acts as a vehicle to reduce the surface tension between the aqueous media and the insoluble active, thereby facilitating the active's maintenance in the aqueous media. The wetting agent may be chosen from available compounds in the art. These can include, for example, sorbitan fatty acid esters, polyoxyethylene sorbitan fatty acid esters, polyoxyethylene fatty acid esters, polyoxyethylene-polyoxypropylene copolymers and block copolymers (also referred to as ethylene oxide/propylene oxide copolymers and block copolymers). It may be especially desirable to substantially minimize or even exclude polyethylene glycol. Thus, the formulation of the invention should preferably contain substantially no polyethylene glycol. It is also preferred to minimize or avoid the presence of propylene glycol, glycerol, and sorbitol as well. The formulations of the invention most preferably contain substantially none of these compounds.

The wetting agent may also be chosen from the broad classes of surfactants, including nonionic, cationic, anionic, and zwitterioninc surfactants known in the industry, some of which may overlap with those compounds mentioned above. Docusate sodium, polysorbate, e.g. polysorbate 80, and polyoxyethylene (40) stearate are useful, either alone or in combination, as wetting agents in the composition of the invention. Other useful wetting agents include the surfactants chosen from the class of ethylene oxide—propylene oxide copolymers (including block copolymers) which enhance flocculation in liquid suspensions, many of which are marketed under the PLURONIC trademark, e.g., PLURONIC® F 127.

A particularly preferred wetting agent for use with the present invention is docusate sodium. Docusate sodium is known chemically as bis(2-ethylhexyl) sodium sulfosuccinate, and also as dioctyl sodium sulfosuccinate and sulfo-butanedioic acid 1,4-bis(2-ethylhexyl) ester, sodium salt. Other suitable docusate compounds and their salts are also within the scope of the invention. Docusate sodium is described as an anionic surfactant which is a white or almost white, wax-like, bitter tasting, plastic solid with a characteristic octanol-like odor. It is hygroscopic and usually available in the form of pellets, flakes or rolls of tissue-thin material. It has now been discovered that the use of docusate sodium, preferably in amounts of about 0.04% or less, results in stable and resuspendable flocculated suspensions. Moreover, it has also been found in regard to certain embodiments that decreasing the concentration of the wetting agent used in conjunction with the active substance to less than about 0.04%, it is further possible to improve flocculation overall. This in turn has further resulted in a noticeable increase in the size of the resulting floccules as well.

A preferred concentration of the wetting agent(s) in the composition of the invention is within the range of about 0.0001%, more preferably about 0.001%, to less than about 2%, and preferably less than about 1%. More perferably, the amount of wetting agent(s) will be in the range of about 0.005% to about 1%, with a range of about 0.01 to about 0.04% being more preferred, and about 0.01 to about 0.03% often being most preferred. When docusate sodium is utilized as the wetting agent, docusate sodium is preferably used in an amount of about 0.001% to about 0.04%, more preferably about 0.005% to about 0.025%, and most preferably about 0.01% to about 0.02%. However, the invention is also intended to include other wetting agents and concomitant amounts thereof in which flocculation overall is achieved, and in particularly preferred embodiments floccule size is actually controlled by increasing or decreasing the concentration of the wetting agent within a certain range.

The size or diameter of the floccules is believed to determine the rate of absorption of the active substance, and thus the therapeutic concentration in the bloodstream as measured against time. Larger flocs tend to expose less surface area of the active drug particles to contact the mucosal surfaces than do smaller flocs. Consequently, with larger flocs there is a lower rate of absorption of the active. Larger flocs are therefore typically desired in applications where it is important that the active substance not be absorbed too readily, i.e., not be in a state where it can be absorbed too quickly by the body. Smaller flocs are generally desired where the rate of absorption is to be accelerated. At the same time, floc size also impacts upon the dosage form in terms of the rate of sedimentation and ease of resuspendability, including the tendency to avoid precipitation in the G.I. tract. Consequently, in a preferred embodiment, the invention provides for the advantageous ability to control floc size so as to provide an optimal balance of several factors contributing to overall pharmaceutical elegance and utility.

It is generally desirable that the floccules in the aqueous suspension composition of the invention have a mean floc size diameter (<90%) up to at least about 12 microns, preferably up to at least about 21 microns, and more preferably up to at least about 23 microns. Stated another way, preferably up to 90% of the flocculated particles in the final formulation should be measured at up to a mean floc size diameter of at least about 12 microns, preferably up to at least about 21 microns, and more preferably up to at least about 23 microns, and even more preferably up to at least about 30 microns. Most preferably, the aqueous suspension will contain flocs whose mean floc size diameter (<90%) is as much as about 40 microns, or even more such as about 50 microns. (“Mean floc size diameter” is to be distinguished from the term “mean particle size diameter,” which as used herein, refers to the diameter of the unagglomerated active particle.)

Other preferred wetting agents along with suitable concentration ranges include the following: ethylene oxide-propylene oxide (EO-PO) copolymers and block copolymers, e.g. PLURONIC® F127, in amounts of about 0.0001 to 1.5%, more preferably about 0.001 to about 0.5%, and even more preferably about 0.005 to about 0.01%.

In certain other embodiments, docusate sodium (in amounts as heretofore described) together with one or more EO-PO copolymers or block copolymers in the above amounts may also be desirable. Especially preferred may be a formulation containing substantially equal amounts of docusate sodium and one or more EO-PO copolymer(s) and block copolymer(s), e.g., those that enhance flocculation such as those available under the PLURONIC trademark, including PLURONIC® F127. As an example, a formulation containing about 0.0001 to about 0.01% of docusate sodium and about 0.0001 to about 0.01% of one or more ethylene oxide/propylene oxide copolymers and block copolymers may be utilized. More preferably, the concentration of the respective wetting agents may be within the range of about 0.001 to about 0.01%. A range of about 0.001 to about 0.005% may also be desirable.

In addition to the foregoing wetting agent(s), the flocculated suspension composition of the invention also desirably contains a suspending agent. The suspending agent is preferably a water-soluble hydrocolloid material. The hydrocolloid material acts to suspend the active substance in the aqueous media. The hydrocolloid material is generally selected from available food-grade and pharmaceutical-grade vegetable and animal sources. It is desirable that this material possess emulsifying and/or thickening properties. In this regard, pharmaceutically acceptable gums and gelatins are preferred. Of these, pharmaceutical grade gums are especially desirable. Gums include, for example, guar gum, carrageenan, gum arabic and xanthan gum. Xanthan gum is particularly desirable for use with active substances such as megestrol acetate. Carbomers (e.g., carboxyvinyl polymers) and derivatives of alginic acid are also desirable and may be utilized.

The suspending agent is preferably utilized at concentration levels of about 0.01% to about 1.0%, with levels of about 0.05% to about 0.5% being more desirable. Even more preferably, amounts within the range of about 0.1% to about 0.3% are used. A concentration range of about 0.2% to about 0.3% for the suspending agent is even more desirable.

In addition to the components just described, the aqueous suspensions in accordance with the present invention may also contain a number of other ingredients. Sweeteners may be utilized as part of the composition to enhance the organoleptic properties of the suspension and to function as taste-masking agents. They may also be chosen to enhance the stability and/or viscosity of the final formulation. The preferred sweetener is sucrose and more preferably, sucrose syrup. Other suitable sweeteners can include, without limitation, saccharide material, and in particular, mono-, di-, tri- and oligosaccharides. Representative examples include glucose and fructose. Other non-limiting examples of suitable sweeteners include mannitol and xylitol. Synthetic sweeteners such as saccharin, sucralose, acesulfame, and aspartame may also be utilized.

The amount of sweetener used in accordance with the present invention can vary based on a number of factors. If the active is particularly bad-tasting, then more sweetener may be used. Generally, the amount of sweetener will range from about 0.01 to about 60%. Preferably, the amount used will be within the range of about 1 to about 20%, with about 2 to about 10% being particularly preferred. An especially desirable embodiment of the present invention will have about 7.6% of sweetener when the active ingredient is megestrol acetate.

Flavorants or flavors may also be used to enhance the organoleptic qualities of the final composition, preferably in synergistic effect with the just described sweetener(s). Any conventional, approved flavorants may be chosen so long as they do not materially affect the physical or chemical attributes of the active or of the resulting suspension. Both natural and synthetic flavorants are contemplated for use herein. Flavorants can therefore include vanilla, strawberry, cherry, grape, lemon, lime, orange, cinnamon and mint such as peppermint and spearmint, and any desired combination thereof. Flavorants will typically be added in amounts of from about 0.005% to about 20%, with about 0.01% to about 5% being especially desirable.

One or more antimicrobial agents or preservatives may also be used to form the aqueous flocculated suspension. Such agents can include, for example, the parabens such as methyl, propyl and butyl paraben, as well as compounds such as sodium benzoate, potassium sorbate, and sodium propionate, to name just a few. Sodium benzoate is particularly preferred. The antimicrobial agent should preferably not interfere with the floccules in the suspension, should be water-soluble, and should not adversely affect the taste or the pH of the final composition. The amount chosen can vary somewhat within a given range. A range of about 0.01% to about 1% is often desirable. Even more preferred is about 0.05 to about 0.5%.

pH modifiers or buffers may also be used to maintain the pH of the final composition within a certain desired range. pH often has a substantial effect on stability, and so the pH chosen should enhance stability of the formulation overall. Thus, the pH modifiers used in accordance with the present invention may be any pharmaceutical grade acid or base which is capable of maintaining the pH within an acceptable range. pH modifiers are generally used within the range of about 0.005 to about 1%, with about 0.01 to about 0.5% being more preferred. To acidify the final formulation, a combination of a weak acid and the salt of a weak acid may be chosen. In this regard, citric acid is particularly useful. Sodium citrate is also desirable. A combination of citric acid and sodium citrate is especially preferred. In addition, any combination of the previously mentioned antimicrobial agents/preservatives together with pH modifiers/buffers which yield a generally suitable ionic strength and pH, and which are pharmaceutically compatible, are suitable for use herein. The functions of an antimicrobial agent and a pH modifier can be obtained through the use of, for example, a mixture of sodium benzoate and citric acid with sodium citrate.

In addition to the foregoing components, the composition of the invention may also contain additional excipients. For example, humectants or other similar types of wetting agents may be used. Also desirable may be compounds useful as anti-foaming agents such as simethicone and others which inhibit or reduce the formation of gas bubbles in the final formulation. FDA-approved colorants may also be chosen to make the formulation more visually palatable. Other viscosity modifiers may also be chosen. These optional ingredients, when included, will generally comprise about 0.01 to about 10% of the aqueous suspension. More desirably, they will comprise about 0.01 to about 0.5%.

The remainder of the compositions according to the invention is preferably water, but may be other potable liquid(s). The potable liquid is utilized in conjunction with the foregoing components so as to provide up to about 100% of the total composition.

Compositions according to the invention may be prepared by any suitable procedure. The following illustrative procedures may be utilized and are often preferred. According to a “two pot” process, a suspending agent (e.g., hydrocolloid material such as xanthan gum) is added (slowly) to purified water under mixing. The resultant batch is then mixed for about an hour to ensure complete hydration of the hydrocolloid matter. Next, one or more preservatives (e.g., sodium benzoate) and pH modifiers (e.g., citric acid and sodium citrate) are added to the batch and the mixture is held to about 25-30° C. with mixing. One or more sweeteners (e.g., sucrose syrup) are then added with mixing. Batch weight is then adjusted, if desired, using purified water. The resultant mixture is referred to as Phase I. In another vessel, the wetting agent(s) (e.g., docusate sodium and/or one or more EO/PO copolymers such as PLURONIC® F 127) is then added to heated purified water (70-75° C.) and thoroughly admixed under high shear mixing. Phase I is then preferably cooled down to 25-30° C. The active (e.g., megestrol acetate) is then admixed into this batch under high shear. The resultant admixture is referred to as Phase II. Phase I is then added to Phase II under mixing and vacuum. The flavorants (e.g., lemon-mint) are then admixed into Phase II as well to produce the final formulation. If need be, formulation weight may be adjusted using purified water.

In another version of the “two pot” process described above, the wetting agent(s) are dissolved in warm water. The resultant solution is then cooled (−25° C.), and the active substance is then added under low or high shear mixing conditions. In a separate container, the hydrocolloid material is prepared with suitable amounts of hot water (65-70° C.) and is then cooled. The remaining flavorants and excipients are then added and the resultant mixture may then be strained to remove any undissolved ingredients. This mixture is then combined with the mixture containing the active substance. The resultant admixture is thoroughly stirred and then passed through a mill. The resultant composition is an aqueous flocculated suspension.

In another embodiment of the invention, a “single pot” method of manufacture may be utilized. Hot purified water (70-75° C.) is transferred to a large pressure vessel. Using a hose and vacuum, a suspending agent (e.g., a hydrocolloid material such as xanthan gum) is preferably added from the bottom of the vessel. High shear mixing is then used to thoroughly hydrate the hydrocolloid material. This mixture then becomes the main phase. While maintaining the temperature of the batch within 55-75° C., one or more wetting agents (e.g., docusate sodium and/or EO/PO copolymers and block copolymers such as PLURONIC® F127) together with any remaining excipients (e.g., preservatives, pH modifiers) are combined under vacuum until all solids are thoroughly dissolved. The mixture is then cooled to between about 25-30° C. while mixing continues. The active (e.g., megestrol acetate) is then added under vacuum and thoroughly mixed into the main phase to ensure that a good dispersion is obtained. Flavorants are then added and mixing continues until a good admixture is obtained. Batch weight is then adjusted, if desired, using purified water.

In yet another embodiment of the invention, the hydrocolloid material, one or more wetting agents and the remaining excipients, and a suitable amount of water are combined with stirring (low or high shear) to thoroughly admix all ingredients. This admixture may be strained or screened. The active substance is then added to this admixture, and is thoroughly dispersed. The resultant mixture is then strained or passed through a colloid mill to yield the aqueous flocculated suspension of the invention. Other means of preparation in addition to any of the foregoing may also be effected by the skilled artisan.

The compositions according to the various embodiments of the invention may be orally administered to a mammal according to a dosing schedule prescribed by an appropriate health official. In a preferred embodiment, the composition is an oral suspension containing pharmaceutically acceptable amounts of megestrol acetate which is suitable for use in humans.

The following examples are intended to highlight certain embodiments of the invention, but should not be construed as limiting the scope thereof.

### EXAMPLE 1

In this example, a megestrol acetate formulation was made using docusate sodium as the wetting agent according to Table 1 below:

TABLE 1

| Ingredient Name | | Percentage by Weight |
|---|---|---|
| Megestrol Acetate | | 40 mg/mL* |
| Docusate Sodium | | 0.01 |
| Xanthan Gum | | 0.25 |
| Sodium Benzoate | | 0.188 |
| Citric Acid | | 0.15 |
| Sodium Citrate | | 0.015 |
| Sucrose Syrup | | 7.6 |
| Artificial Lemon-Mint Flavor | | 0.045 |
| Simethicone Emulsion | | 0.016 |
| Purified Water | | QS to 100% |
| Floc Size Data, microns: | 10% < | 9 |
| | 50% < | 22 |
| | 90% < | 38 |

*Weight/Volume

The formulation according to Table 1 was a flocculated suspension which was storage stable, and re-suspendable upon light to moderate shaking.

### EXAMPLE 2

In this example, a megestrol acetate formulation was made using PLURONIC® F127 as the wetting agent according to Table 2 below.

TABLE 2

| Ingredient Name | | Percentage by Weight |
|---|---|---|
| Megestrol Acetate | | 40 mg/mL* |
| PLURONIC F127 | | 0.01 |
| Xanthan Gum | | 0.25 |
| Sodium Benzoate | | 0.188 |
| Citric Acid | | 0.15 |
| Sodium Citrate | | 0.015 |
| Sucrose Syrup | | 7.6 |
| Artificial Lemon-Mint Flavor | | 0.045 |
| Simethicone Emulsion | | 0.016 |
| Purified Water | | QS to 100% |
| Floc Size Data, microns: | 10% | 7 |
| | 50% | 21 |
| | 90% | 36 |

*Weight/Volume

The formulation according to Table 2 was a flocculated suspension which was storage stable, and re-suspendable upon light to moderate shaking.

### EXAMPLE 3

In this example, a megestrol acetate formulation was made using a combination of docusate sodium and PLURONIC® F127 as the wetting agents according to Table 3 below.

TABLE 3

| Ingredient Name | | Percentage by Weight |
|---|---|---|
| Megestrol Acetate | | 40 mg/mL* |
| Docusate Sodium | | 0.005 |
| PLURONIC F127 | | 0.005 |
| Xanthan Gum | | 0.25 |
| Sodium Benzoate | | 0.188 |
| Citric Acid | | 0.15 |
| Sodium Citrate | | 0.015 |
| Sucrose Syrup | | 7.6 |
| Artificial Lemon-Mint Flavor | | 0.045 |
| Simethicone Emulsion | | 0.016 |
| Purified Water | | QS to 100% |
| Floc Size Data, microns: | 10% | 5 |
| | 50% | 16 |
| | 90% | 32 |

*Weight/Volume

The formulation according to Table 3 was a flocculated suspension which was storage stable, and re-suspendable upon light to moderate shaking.

### EXAMPLE 4

(Method for the Determination of Floccule Size)

This example provides a suitable method for measuring the size of flocs in a liquid suspension. Equipment needed includes a Malvern Mastersizer X or equivalent, a 100 mm lens, a mixed cell adapter, and scintillation vials (20 mL) or equivalent. A sample cell is filled and mixed with deaerated purified water without incorporating air. 1.0 gram of the sample suspension is then added to a vial, and its weight is adjusted using 10.0 grams of the deaerated purified water. The vial is then covered and gently shaken until uniform. The suspension is then transferred to the sample cell using a dropper to obtain an obscuration level of about 15-30%. The sample cell is then mixed for about 2 minutes before taking a measurement. At least two readings, and preferably no more than three per sample, and then recorded and reported as 10%, 50% and 90% undersize. At least two sample readings should be within 10% of each other in order to be acceptable. Next, two more samples (for a total of three sample preparations) are prepared and read as described above, and the average of six readings is then recorded (rounded up to the next whole number).

The foregoing description is illustrative of exemplary embodiments which achieve the objects, features and advantages of the present invention. It should be apparent that many changes, modifications, and substitutions may be made to the described embodiments without departing from the spirit or scope of the invention. The invention is not to be considered as limited by the foregoing description or embodiments, but is only limited by the construed scope of the appended claims.

What is claimed is:

1. A composition comprising at least one insoluble active substance together with at least one wetting agent, wherein the concentration of said wetting agent is sufficient to form a stable, flocculated suspension of said active substance, wherein said composition contains substantially no polyethylene glycol, propylene glycol, sorbitol or glycerol.

2. The composition of claim 1, wherein said active substance is megestrol acetate and said wetting agent is at least one member selected from the group consisting of nonionic, cationic, anionic, and zwitterionic surfactants.

3. The composition of claim 1, wherein the concentration of said wetting agent is within the range of about 0.0001% to about 1.5% w/w.

4. The composition of claim 1, wherein the concentration of said wetting agent is within the range of about 0.0005% to about 1% w/w.

5. The composition of claim 1, wherein the concentration of said active substance is within the range of about 0.1% to about 25% w/v.

6. The composition of claim 1, wherein the concentration of said active substance is within the range of about 1 to about 10% w/v.

7. The composition of claim 1, wherein the concentration of said active substance is about 4% w/v.

8. The composition of claim 1, further comprising at least one suspending agent.

9. The composition of claim 8, wherein said suspending agent is present at a concentration within the range of about 0.01 to about 1.0% w/w.

10. The composition of claim 8, wherein said suspending agent is present at a concentration of within the range of about 0.1% to about 0.3% w/w.

11. The composition of claim 8, wherein said suspending agent is at least one hydrocolloid material.

12. The composition of claim 11, wherein said hydrocolloid material is at least one member selected from the group consisting of pharmaceutically acceptable gums.

13. The composition of claim 1, wherein said composition comprises at least two wetting agents.

14. The composition of claim 1, wherein said wetting agent is at least one member selected from the group consisting of docusate sodium and ethylene oxide/propylene oxide copolymers and block copolymers.

15. The composition of claim 14, wherein the concentration of said wetting agents is within the range of about 0.0001 to about 0.04%.

16. The composition of claim 15, wherein the concentration of said wetting agents is within the range of about 0.001 to about 0.02%.

17. The composition of claim 13, wherein said composition further comprises at least one suspending agent.

18. A method for forming an aqueous flocculated suspension containing an insoluble micronized active substance together with a wetting agent to form a stable, resuspendable flocculated suspension of said active substance, which comprises adding said wetting agent in an amount below which the floccule size of said active substance in said suspension starts to increase, wherein substantially no polyethylene glycol, propylene glycol, sorbitol or glycerol is included in said suspension.

19. The method of claim 18, wherein said active substance is megestrol acetate.

20. The method of claim 18, wherein said wetting agent is docusate sodium.

21. The method of claim 20, which comprises adding said docusate sodium in an amount of about 0.001 to about 0.03% w/w.

22. The method of claim 20, which comprises adding said docusate sodium in an amount of about 0.005% to about 0.01% w/w.

23. The method of claim 18, wherein said flocculated suspension comprises floccules having a mean floc size diameter of at least about 12 microns.

24. The method of claim 18, wherein said flocculated suspension comprises floccules having a mean floc size diameter of about 12 to about 50 microns.

25. The method of claim 18, wherein said flocculated suspension comprises floccules having a mean floc size diameter of about 23 to about 40 microns.

26. An oral pharmaceutical composition, comprising:
a) about 0.5 to about 10% w/v of megestrol acetate;
b) about 0.001 to about 2% w/w of at least one wetting agent selected from the group consisting of docusate sodium and ethylene oxide/propylene oxide copolymers and block copolymers; and
c) about 0.05 to about 0.5% w/w of at least one suspending agent, wherein said composition contains substantially no polyethylene glycol, propylene glycol, sorbitol or glycerol.

27. The composition of claim 26, comprising about 0.005 to about 0.04% w/w of docusate sodium.

28. The composition of claim 26, wherein said suspending agent is a pharmaceutical grade gum.

29. The composition of claim 27, comprising about 0.005 to about 0.02% w/w of docusate sodium.

30. The composition of claim 27, comprising about 0.005 to about 0.02% of at least one member selected from the group of ethylene oxide propylene oxide copolymers and block copolymers.

31. The composition of claim 30, wherein said suspending agent is present in said composition in an amount of from about 0.1 to about 0.3% w/w.

32. The composition of claim 29, wherein said composition contains floccules having a mean floc size diameter of at least about 12 microns.

33. The composition of claim 32, wherein said composition contains floccules having a mean floc size diameter of at least about 21 microns.

34. An oral composition, comprising about 1 to about 8% w/v of megestrol acetate, wetting agents consisting essentially of about 0.005 to about 1% w/w of docusate sodium and about 0.005 to about 1% w/w of at least one ethylene oxide propylene oxide copolymer; and further comprising from about 0.1 to about 0.3% w/w of at least one hydrocolloid material.

35. The composition of claim 34, wherein said composition is in the form of an aqueous flocculated suspension which is storage stable for at least about 3 months.

36. The composition of claim 34, wherein said composition is storage stable for at least about 12 months.

37. The composition of claim 34, said composition containing substantially no polyethylene glycol.

38. The composition of claim 34, said composition containing substantially no propylene glycol, glycerol or sorbitol.

39. A method of forming an oral pharmaceutical composition, comprising:
combining a first portion of a pharmaceutical grade gum and water in a first vessel;
combining a second portion of megestrol acetate and at least one wetting agent in a second vessel;
combining the contents of said first vessel with the contents of said second vessel, wherein said wetting agent is at least one member selected from the group consisting of docusate sodium and ethylene oxide/propylene oxide copolymers and block copolymers, and further wherein said composition contains substantially no polyethylene glycol, propylene glycol, sorbitol or glycerol.

40. A method of forming an oral pharmaceutical composition, comprising:
admixing megestrol acetate and at least one wetting agent, said wetting agent consisting essentially of at least one member selected from the group of docusate sodium and ethylene oxide/propylene oxide copolymers, wherein said composition contains substantially no polyethylene glycol, propylene glycol, sorbitol or glycerol.

41. The composition of claim 1, wherein said wetting agent is within the range of about 0.001 to about 0.05% w/w.

42. An oral composition, comprising about 1 to about 8% w/v of megestrol acetate, a wetting agent consisting essentially of docusate sodium; and further comprising from about 0.1 to about 0.3% w/w of at least one hydrocolloid material.

43. An oral composition, comprising about 1 to about 8% w/v of megestrol acetate, a wetting agent consisting essentially of an ethylene oxide/propylene oxide block copolymer; and further comprising from about 0.1 to about 0.3% w/w of at least one hydrocolloid material.

44. The composition of claim 43, wherein said block copolymer is PLURONIC F 127.

45. The composition of claim 1, further comprising an anti-foaming agent.

46. The composition of claim 26, further comprising an anti-foaming agent.
